(19) United States
(12) Patent Application Publication
(10) Pub. No.: US 2006/0099149 A1
(43) Pub. Date: May 11, 2006

(54) Metered dose inhaler product

(75) Inventor: Kachan Nagin Patel, Surrey (GB)

Correspondence Address: HESLIN ROTHENBERG FARLEY & MESITI PC, 5 COLUMBIA CIRCLE, ALBANY, NY, 12203, US

(73) Assignee: Karib Kemi-Pharm Limited, Surrey (GB)

(21) Appl. No.: 11/281,327

(22) Filed: Nov. 17, 2005

Related U.S. Application Data
(63) Continuation of application No. PCT/GB04/02190, filed on May 21, 2004.

(30) Foreign Application Priority Data
May 21, 2003 (GB) GB 0311701.7

(51) Int. Cl. A61L 9/04; A61M 11/00

(52) U.S. Cl. 424/45; 128/200.23

(57) ABSTRACT

The present invention relates to the provision and use of pressurised metered dose inhalers (MDIs) for the effective administration of pharmaceutical aerosol formulations. Such formulations comprise a drug, a propellant comprising one of either 1,1,1,2-tetrafluoroethane (HFA 134a) or 1,1,1,2,3,3,3-heptafluoropropane (HFA227) or a mixture thereof, a cosolvent having a higher polarity than HFA 134a or HFA227, and a surfactant in an amount at least 0.01% by weight of said formulation. Such MDIs comprise a canister.

### CROSS REFERENCE TO RELATED APPLICATIONS

This application is a Continuation application of PCT Application Serial Number PCT/GB2004/002190, filed May 21, 2004, published in English on Dec. 2, 2004, Publication Number WO 2004/103339, which PCT application claims priority from Great Britain application GB 0311701.7, filed May 21, 2003, which are hereby incorporated by reference in their entirety.

### FIELD OF THE INVENTION

The present invention is principally directed towards the provision and use of pressurised metered dose inhalers (MDIs) for the effective administration of pharmaceutical aerosol formulations; in particular formulations including steroids such as beclamethasone dipropionate, fluticasone propionate, salbutamol sulphate or budesonide, and, more particularly, formulations including hydrofluoroalkane (HFA) propellants.

### BACKGROUND OF THE INVENTION

Pressurised MDIs are well known as effective delivery devices for the administration of pharmaceutical products to the respiratory tract by inhalation. Historically, chlorofluorocarbons (CFCs) such as monofluorotrichloromethane or dichlorodifluoromethane have been used as propellants for drug administration by MDIs. However, owing to the detrimental effects of CFCs on the atmospheric ozone layer, the use of CFCs is gradually being phased out.

Efforts have accordingly been made to identify a suitable alternative non-CFC propellant for use in MDIs. Research in this area has focused on hydrofluoroalkanes (HFAs), and in particular on 1,1,1,2-tetrafluoroethane (HFA 134a) and 1,1,1,2,3,3,3-heptafluoropropane (HFA227). Each of HFA 134a and HFA227 has been widely acknowledged as a suitable alternative to CFC propellants for use in drug administration.

To this end, EP-B-0372777 to Riker Laboratories Inc. describes the use of HFA134a as a propellant for metered dose inhalers. Specifically, EP-B-0372777 describes the use of HFA134a propellant in metered dose inhalers for the aerosol administration of salbutamol, beclamethasone dipropionate, disodium cromoglycate, pirbuterol, isoprenaline, adrenaline, rimiterol or ipratropium bromide.

However, whilst HFAs have been demonstrated to be safe for inhalation and hence suitable for use as propellants in MDIs, problems have been encountered in formulating compositions including HFA propellants. More particularly, it has been found that other formulation excipients, specifically surfactants such as sorbitan trioleate and oleic acid, are inadequately soluble in HFAs. Solubilised surfactants assist in the preparation of stable and effective aerosol formulations, and are particularly important in suspension formulations where they serve to improve drug particle dispersion. In order to improve the solubilisation of surfactants in HFA-containing aerosol formulations, EP-B-0372777 mentions the addition to the formulation of a co-solvent having a higher polarity than HFA134a. The co-solvent serves to solubilise surfactant in the composition, hence enabling the use of increased amounts of surfactant which will improve the stability and efficacy of the aerosol formulation.

Whilst surfactants such as oleic acid and sorbitan trioleate serve to improve formulation stability and efficacy, the use of such surfactants in conjunction with the aluminium containers typically used to store and dispense aerosol formulations has proved problematic. Reactions between the oleate surfactant and the aluminium walls of the container result in the formation over time of metal oleates, causing product degradation. In order to address this problem, attempts have been made to reduce the quantity of surfactant used in the formulation, so as to minimise the rate of formation of oleates. However, this is not a preferred solution to the problem, as a reduction in surfactant quantity has an adverse effect both on control of mist generation by the MDI on dispersal of the formulation, and on the lubrication of the dispenser and valve mechanism used to disperse the formulation. Impaired valve lubrication, resulting from diminished surfactant levels, may give rise to excess friction between the working parts of the valve, which may damage the valve and/or may generate particulate matter that will contaminate the formulation.

It is therefore an object of the present invention to enable the production and use of an aerosol formulation including a medicament, HFA propellant and sufficient quantities of a surfactant, avoiding or ameliorating the problems described above.

### SUMMARY OF THE INVENTION

According to one aspect of the present invention therefore, there is provided an aerosol formulation comprising a drug, a propellant comprising one of either 1,1,1,2-tetrafluoroethane (HFA 134a) or 1,1,1,2,3,3,3-heptafluoropropane (HFA227) or a mixture thereof, a cosolvent having a higher polarity than HFA134a or HFA227, and a surfactant in an amount at least 0.01% by weight of said formulation.

### BRIEF DESCRIPTION OF DRAWINGS

FIG. 1 is a cross sectional view of a metered dose inhaler including sleeve, canister and valve arrangement according to the prior art;

FIG. 2 is an enlarged sectional view of the actuator of the metered dose inhaler of FIG. 1;

FIG. 3 is a cross sectional view along the line III-III of FIG. 2;

FIG. 4 is an enlarged sectional view of the actuator of a metered dose inhaler according to the present invention;

FIG. 5 is a cross sectional view along the lines V-V of FIG. 4.

### DETAILED DESCRIPTION OF THE INVENTION

In some useful embodiments of the invention, the surfactant may be capable of reacting with a metal such as aluminium so as to form salts of the free metal. Such salts may constitute undesired contaminants in aerosol formulations. For example, the surfactant may comprise oleic acid, which is capable of reacting with aluminium to form aluminium oleate. Alternatively, said surfactant may comprise ethyl oleate, sorbitan trioleate, isopropyl myristate, or other such surfactants.

Said drug may be beclamethasone dipropionate, salbutamol sulphate, fluticasone propionate or budesonide. These are preferred but non-limiting examples.

Said cosolvent may be an alcohol, such as ethanol or isopropanol, or propylene glycol. However, any suitable cosolvent may be used.

Suitably, said formulation may comprise HFA 134a and not HFA227.

Suitably, said surfactant may be present in an amount up to about 0.1%, such as up to about 0.089% by weight of the formulation. More preferably, said surfactant may be present in an amount between 0.01-0.02% by weight, even more preferably about 0.015% by weight, of said formulation. This level of surfactant is sufficient to ensure product stability and proper lubrication of the dispenser and valve mechanism.

Advantageously, the wt/wt ratio of said surfactant to said drug may be in the range 0.05-0.5, such as in the range 0.1-0.3, such as about 0.2.

Typically, said surfactant and said drug together may constitute 0.03-0.5% by weight, such as 0.05-0.1% by weight, such as about 0.09% by weight, of said formulation.

Advantageously, said drug may be present in an amount between about 0.01-0.5% by weight, preferably between about 0.014-0.445% by weight.

Preferably, the wt/wt ratio of said cosolvent to said propellant may be in the range 0.09-0.1.

Said cosolvent may be present in an amount up to about 25% by weight of the formulation, such as in an amount between 0.8-25% by weight of the formulation.

In preferred embodiments of the invention, said formulation may comprise 0.015-0.089% by weight of said surfactant; 0.014-0.445% by weight of said drug; 0.856-25.68% by weight of said cosolvent; and at least 73.786% by weight of 1, 1,1,2-tetrafluoroethane.

Said formulation may comprise one or more additional excipients or additives.

Preferably, said formulation may be a solution formulation. Alternatively, said formulation may be a suspension formulation. Advantageously, said formulation may be adapted for airborne dispersion and inhalation by a patient.

According to another aspect of the present invention, there is provided a method for producing a formulation in accordance with the invention, comprising the steps of mixing said cosolvent, said surfactant, and said drug, and thereafter adding said 1,1,1,2-tetrafluoroethane. Preferably, said cosolvent and said surfactant may be mixed together prior to addition of said drug, in order to improve the dispersion of the drug in the formulation.

Advantageously, said method may be carried out at a temperature greater than 4° C.; preferably a temperature greater than 8° C. Said method may be carried out at a temperature no greater than 30° C. More particularly, said method may be carried out at ambient temperature or at a temperature marginally above or below ambient temperature, such as up to 10° C. above or below ambient temperature. In especially preferred embodiments, said method may be carried out at a temperature between 20-25° C.

Suitably, said steps of mixing said surfactant, said cosolvent and said drug may be carried out in one or more non-pressurised vessels or in one or more moderately closed vessels. Prior to addition of said propellant, the mixture comprising said drug, said surfactant and said cosolvent may preferably be held in a sealable vessel; and said vessel may preferably be sealed by a valve prior to addition of said propellant through said valve. Said vessel may, for example, comprise an aluminium canister, such as an aluminium canister in accordance with the invention as set out hereinbelow. Said valve may comprise a metering valve.

Suitably, said propellant may be added to said vessel under a degree of pressure, such as under a pressure of up to 15 bar, such as 10-12 bar. The internal vessel pressure following addition of said propellant may be 3-6 bar, such as about 4.5-5.5 bar.

Advantageously, said drug, cosolvent and surfactant may be mixed for up to one hour, such as for 15-45 minutes, such as for about 30 minutes, prior to addition of said propellant.

According to another aspect of the present invention, there is provided a metered dose inhaler comprising a canister which contains a formulation in accordance with the present invention. Suitably, said canister may contain approximately 15-20 g, such as about 17-18 g, of said formulation. Preferably, said inhaler may be arranged to dispense 10-300 mcg, such as 20-200 mcg or 25-125 mcg, of said drug on each actuation of the inhaler.

Owing to the relatively high concentration of surfactant in the formulation, the use of an aluminium canister for containing the formulation may result in the undesirable formation of impurities in the formulation, such as metal oleates (where oleic acid is used as surfactant). The inventors have however found that this problem can be alleviated through the provision and use of an aluminium canister that is anodised on its interior surfaces.

According to yet another aspect of the present invention therefore, there is provided an aluminium canister for use in a metered dose inhaler, which canister is anodised on the interior surfaces thereof.

According to yet another aspect of the present invention, there is provided an aluminium canister in accordance with the invention, which canister contains a quantity of the formulation of the present invention. Suitably, said canister may contain approximately 15-20 g, such as about 17-18 g, of said formulation.

According to yet another aspect of the present invention, there is provided a method for manufacturing an anodised aluminium canister, comprising the steps of providing an aluminium canister, polishing the interior surfaces of the canister, and thereafter anodising said interior surfaces of the canister. Said step of polishing the interior surfaces of the canister serves to provide a smooth surface for anodisation. Said polishing step may involve placing a granular polishing material, such as a powder or a plurality of small balls, into said canister, and agitating said canister and/or said polishing material such that the interior surfaces of the canister are polished by the polishing material. Optionally, a soap solution, such as a mild soap solution, may be added to said granular polishing material such as to clean the interior surfaces of the canister during polishing. Additionally or alternatively, said step of polishing the interior surfaces of the canister may involve electro-polishing. Electro-polishing is a technique familiar to the man skilled in the art, commonly used for the removal of surface matter from alloys such as stainless steel. When used for polishing the interior surfaces of said canister, the technique will involve the construction of an electrolytic circuit utilising the interior surfaces of the canister as an anode and a suitable conductor as a cathode; immersing the anode and cathode in an electrolyte, typically an acidic electrolyte, and transmitting current through the circuit such as to permit electrolysis. The result of this process will be the removal of surface matter from the anode, thereby “micro-polishing” the surface of the anode.

Still further according to the present invention there is also provided a metered dose inhaler comprising a canister, preferably but not necessarily formed from anodised aluminium, which contains a formulation in accordance with the present invention and which further comprises a metered dose valve formed of polybutylene terephthalate (PBT). Typically, said valve may comprise a metering chamber, an upper stem, a lower stem and a three slot housing formed from PBT. Said valve may comprise gaskets formed from chloroprene. PBT is effectively inert to reaction with HFA propellants, and thus the use of this material for the manufacture of the metering valve results in an improved MDI with an increased shelf life.

According to a further aspect of the present invention there is provided a method for manufacturing a metered dose inhaler comprising a canister adapted for containing a formulation, said canister having a metered valve and an internal receiver for cooperative engagement with said metered valve, which receiver comprises an elongate expansion chamber including an emission orifice, which chamber is arranged to receive metered doses of said formulation from said metered valve and to emit said doses via said emission orifice in a spray for spraying from the inhaler; said method comprising the step of selecting the volume of said expansion chamber and/or the size and/or location of said emission orifice, such that each metered dose of said formulation is sprayed from said inhaler substantially according to a predetermined spray pattern.

Preferably, said predetermined spray pattern may be the spray pattern that is usually obtained on dispensing a formulation comprising a different propellant, such as a CFC propellant, from a conventional inhaler. Suitable values for the volume of said expansion chamber and/or the size and/or location of said emission orifice may be readily identified and selected by trial and error, by varying the receiver design and monitoring the emitted spray pattern from the inhaler.

Alternatively, suitable values for the volume of said expansion chamber and/or the size and/or location of said emission orifice may be identified by providing a metered dose inhaler having a receiver and a metered valve, which inhaler is capable of dispensing metered doses of a different formulation according to said predetermined spray pattern; measuring or noting the volume of the expansion chamber and/or the size and/or location of the emission orifice of said inhaler; measuring or noting the internal pressure of said different formulation in said inhaler; calculating the ratio of said chamber volume and/or the size and/or location of said emission orifice to said internal pressure; measuring or noting the internal pressure of a formulation according to the present invention in said inhaler; and calculating the changes required to said expansion chamber volume and/or the size and/or location of said emission orifice in order to maintain said ratio.

The volume of the expansion chamber, and the size and location of the emission orifice, are each factors which affect the spray pattern produced by the inhaler. In particular, adjustments to the location of the emission orifice along the length of the elongate expansion chamber will vary the product flow path between the metered valve and the emission orifice, hence affecting the velocity of the formulation dispensed from the orifice. Meanwhile, alterations in the volume of the expansion chamber will affect the pressure of the metered dose within the chamber, hence altering the spray pattern produced on dispensation.

Preferably, at a propellant pressure of between 70 and 85 psi the emission orifice may be disposed at a position between 65% and 75%, usually between 70% and 74%, along the length of said chamber.

Preferably, the chamber length may be between 5.95 mm and 18.95 mm in total length, and usually between 8 mm and 12 mm, for a propellant having a pressure of between 70 and 85 psi.

Still further according to the present invention there is provided a metered dose inhaler produced in accordance with the method of the invention, which metered dose inhaler is adapted for providing an output spray pattern of the formulation of the present invention which corresponds to the output spray pattern of a formulation from a conventional MDI, which formulation corresponds to the formulation of the present invention but includes a CFC propellant in place of the HFA propellant.

Usually, the emission orifice may have a diameter of between 0.2 mm and 0.3 mm.

In addition, the expansion chamber may also be tapered in a longitudinal direction such that its cross sectional area at a remote internal end of such expansion chamber is less than 50% of the cross sectional area of the largest cross sectional area of such expansion chamber. Preferably, this remote end may be less than 30% of such cross sectional area. It is usual that the expansion chamber may be substantially cylindrical at a first end which cylindrical section extends for at least 25%, preferably 25-30%, of the overall length of the expansion chamber. In such embodiments, the chamber comprises a tapered section comprising an inclined inner wall of said chamber, which wall is inclined at an angular range of between 5° and 35°, such as between 14° and 16°, relative to an axis of said cylindrical chamber, said wall usually being flat, and usually extending between 65% and 75% of the total length of the chamber.

There will now be described, by way of example only, a preferred embodiment of the present invention with reference to the accompanying illustrative drawings.

The batch manufacturing formula for producing a preferred formulation in accordance with the present invention is as follows:

| | | |
|---|---|---|
| Beclamethasone dipropionate BP | 132.60 | g |
| (micronised) | | |
| Oleic acid BP | 26.52 | g |
| Ethanol BP | 15.300 | kg |
| Propellant HFA134a | 163.200 | kg |

In order to produce the formulation, the oleic acid and ethanol are mixed together at an ambient temperature of 23° C. and relative humidity of 40%. After mixing, a quantity of beclamethasone dipropionate is added. This sequence of steps ensures satisfactory dissolution of the drug without undesirable conglomeration. A quantity of the resulting mixture is then dispensed into an anodised aluminium canister, and a metered valve is placed on the canister to close the canister. The canister is crimped. The propellant is then charged into the canister under pressure. The internal pressure of the sealed canister, after addition of the propellant, is approximately 4.0 bar.

Anodised aluminium canisters for use in the invention are produced in accordance with the following method. Conventional (non-anodised) aluminium canisters are placed in a vibrating bowl containing approximately 30 kg of small stainless steel balls having an average diameter of 4-5 mm, together with a mild soap solution. The vibration of the balls over the surfaces of the canister in the presence of the soap solution cleans and polishes the surfaces, rendering them substantially free of particulate matter. The canister is then electro-polished so as to smooth the surfaces on a microscopic level, removing substantially all grooves and cavities.

The polished canister is then anodised using conventional anodisation techniques, so as to create a protective coating of aluminium oxide which is resistant to reaction with oleate surfactants.

The specific method of manufacture of the anodised containers by combined use of a ball polishing technique, electro-polishing and subsequent anodisation provides significant advantages by not only providing an appropriate anodised container but ensuring removal of all small aluminium particulate material which can become detached from the surface of the container even after anodisation so as to expose non-anodised aluminium surfaces which can still react with the oleic acid. Effective anodisation of the container enables the use of oleic acid surfactant at increased concentrations of 0.01% or more.

It has been found that the use of hydrofluoroalkanes (HFAs) as propellant results in higher inherent internal pressures within the charged metered dose inhaler container as compared to the use of CFC propellants due to their higher vapour pressures. Whereas charging of the containers with conventional CFC propellants will normally result in an inherent pressure in the container of 45 psi-60 psi, a comparable internal pressure of a charge container utilising such HFA propellants will be between 70 psi and 85 psi. The result of this is that conventional actuator nozzle designs for metered dose inhalers utilising HFA propellants will result in a significant increase in efficiency of delivery of the medicament in the spray emitted by such dose inhalers. This will result in an increased delivery of the medicament product per dosage. This increase in efficiency is further enhanced by the high concentration of the surfactant, oleic acid, which also ensures a very fine mist generation with controlled globule size in the atomised spray emitted, whereby the finer size of droplets increases the resultant travel distance ensuring a better transference of dosage of the medicament. Whilst such efficiency has certain advantages, the inherent variance in dosage effected by the new propellant as compared to a CFC propellant, will necessitate further clinical approval of such metered dose inhalers with the appropriate regulatory authority. In order to avoid the drawbacks of further medical testing and approval, the present invention further incorporates a modified design of metered dose inhaler to effect a retarded velocity of the emitted pharmaceutical composition therefrom so as to provide a comparable aerosol spray pattern to that currently approved for the emission of beclamethasone dipropionate metered dose inhalers and achieved by use of lower pressure CFC propellants. This is effected by a mechanical modification of the metered dose inhaler, particularly in a receiver core of the adapter of such metered dose inhaler, to effect a variance in the pressure of the emitted aerosol medicament.

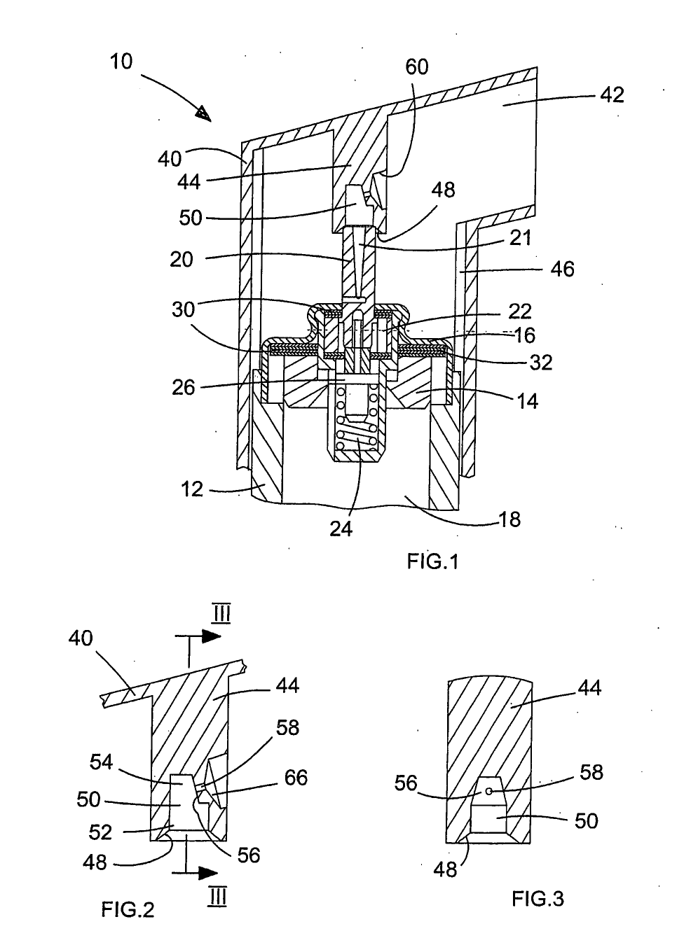

Referring now to FIG. 1, a conventional metered dose inhaler (10) is shown, comprising a drawn aluminium cylindrical container (12), and a metered valve (indicated generally by reference number 14) which is crimped by an appropriate ferrule (16) into sealed engagement with the container (12). A pharmaceutical composition can be contained within the cylindrical aperture (18) of this sealed container (12). The valve itself comprises a hollow cylindrical upper stem (20) projecting externally from the container, which is displaceable into fluid communication with a metering chamber (22) when displaced inwardly of the container (12) against the resilient biasing force of a spring member (24). Here, a lower stem (26), which is engaged with the upper stem (20), effects engagement and is biased by the spring member (24) to the unactuated (and sealed) position shown in FIG. 1 as is conventional for such metered valves. An array of stem gaskets (30) and sealing gaskets (32) maintain the sealed integrity of the valve (14) and container (12).

The specific type of metered valve utilised within the MDI of the present invention is not important to operation of the current invention, and may utilise any known and existing type of metered valve used within the field of metered dose inhalers. Such valve operation need not be described herein in any great detail. However, it is preferred to utilise a polybutylene terephthalate (PBT) metered valve in which the metering chamber (22), the upper stem (20), the lower stem (26) and three slot housing is made out of this polymer, whilst the gaskets (30, 31) are formed of chloroprene.

The applicant has established that this particular material, PBT, is substantially inert with respect to MDI formulations of the type hereinbefore described and, specifically, MDI formulations including HFA propellants. In contrast, polypropylene which is commonly used for the manufacture of metered valves does not enjoy this advantage.

Effectively, inward displacement of the upper stem (20) activates the metered valve by allowing the pressurised emission of a pre-determined volume of the pharmaceutical composition from the container (12) via fluid communication between a hollow core (21) of the outer stem (20) and the metering chamber (22), and allowing the contents of the metering chamber (22) to be emitted under pressure through such hollow core (22).

Operation of the metered dose inhaler and controlled dispersal of the pharmaceutical product is achieved by use of a conventional plastic adapter (40) which effectively forms a cylindrical sleeve encompassing the major portion of the cylindrical container (12). This adaptor (40) has disposed at an upper end thereof an appropriate mouthpiece (42) which presents an opening communicating the interior of such adaptor (40). The adaptor (40) is further provided, on an inner surface thereof with a receiver (44) which engages with and effects actuation of the upper stem (20) of the valve (14). The inner surface of the adapter (40) is also provided with a plurality of ribs (46) for ensuring concentric engagement of the sleeve with the container and for maintaining the receiver in correct engagement with the upper stem (20) as shown in FIG. 1.

The receiver, in an unactuated position as shown in FIG. 1, is in abutting engagement with this upper stem (20) which fits snugly within an initial conical opening (48) of the receiver, whereby this conical opening arrangement of the receiver provides a tapered lead-in surface which ensures overlap of the upper stem (20) within the receiver. The receiver (44) further provides an expansion chamber (50) which is thus maintained in fluid communication with the hollow core (21) interior of the stem (20).

Referring now to FIG. 2, which shows an enlarged view of the receiver (44), it can be seen that the expansion chamber (50) is substantially cylindrical, having a lower portion (52) of a first cylindrical diameter less than the outer diameter of the conical opening (48), which conical opening (48) tapers towards the inner wall of the chamber (50). An upper section (54) of the chamber (50) is then tapered by the ingress of a substantially flat surface (56) which is inclined, at an angle of between 5° and 35°, and preferably 15°, relative to an axis of the cylindrical chamber (50). Extending perpendicularly through this flat surface (56) is a cylindrical emission orifice (58) which is also inclined relative to the axis of the cylindrical chamber (50). This emission orifice (58) then opens out into an emission chamber (60) which is coaxial with an axis of the emission orifice and inclined to the axis of the expansion chamber (50) as shown in FIG. 2. The angle of inclination of this emission orifice and emission chamber serves to effect a change of direction of the pharmaceutical composition emitted from the container (12) so as to be emitted from the receiver (44) in a direction aligned with the mouthpiece (42).

In operation, a user will grasp the adaptor (40) and effect displacement of the container (12) relative thereto so as to displace the receiver (44) towards the valve and (14) effecting an inward displacement of the upper nozzle (20) (by virtue of engagement between the nozzle (20) and the conical surface (48)) so as to achieve fluid communication, through the nozzle (20), between the metering chamber (22) and the expansion chamber (50). The pressured pharmaceutical composition then passes into the chamber (50) where it undergoes expansion and deflection before being emitted through the emission orifice (58) and the emission chamber (60) as an aerosol spray so as to be delivered to the user.

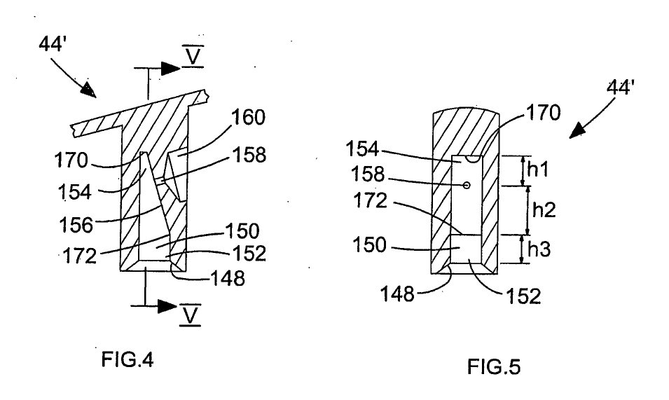

Referring now to FIGS. 4 and 5, there is shown an improved receiver (44′) according to the present invention. The basic receiver design (44′) corresponds to that of existing receiver designs and as shown in FIGS. 1 through 3, whereby like portions of the modified design (44′) will be identified with like reference numbers identifying similar features of the prior art but clarified by use of the prefix “1”. Thus the receiver (44′) also comprises a cylindrical body with a conical opening (148), expansion chamber (150) an inclined flat surface (156) again inclined at an angle between 5° and 30° (preferably 15°) relative to the chamber (150) axis, (which tapers the chamber (150)), an emission orifice (158) and an emission chamber (160). Again, the expansion chamber (150) has a conical lower portion (152) and a tapered upper portion (154). This tapered upper portion results in a gradual decrease in the cross sectional area of the expansion chamber so that at an inner end surface of the expansion chamber the cross section is less than 50% of the maximum cross section of such chamber (and more usually will be less than 30% of such maximum cross sectional area).

However, the improved receiver (44′) is of increased size in comparison to that of the prior art device shown in FIG. 2 and FIG. 3 so as to provide an expansion chamber (150) having an increased volume to allow greater expansion of the pressurised pharmaceutical composition that is injected therein by operation of the MDI metering valve. This greater expansion of the pharmaceutical product effected by the larger expansion chamber (150) serves to reduce such pressure of the emitted pharmaceutical composition, whereby the higher pressure resultant from use of HFA propellants (as previously described) is reduced, such that the emitted dose or respirable fraction of the invention is commensurate with the emitted dose or respirable fraction of pharmaceutical products utilising CFC propellants. This is achieved by varying the expansion chamber design, as compared to the prior art, so that the emitted spray patterns of the MDI using HFA propellant is similar to that produced by MDIs of the prior art utilising CFC propellants at low pressure.

In particular, and with reference to FIG. 5, the expansion chamber (150) can be considered to comprise four separate sections. These are the conical opening (148), the lower cylindrical section (152) which has a height h3 as shown in FIG. 5, and an upper section (154) which is tapered by an inclined flat surface (156) relative to the axis of the conical section (150). This upper section (154) comprises two distinct distances h1, extending between the centre of the emission orifice (58) and an innermost surface (170) of the receiver chamber (150), and a second length h2 extending between the centre of the emission orifice (158) and a delineating step (172) between the inclined surface (156) and the conical lower portion (152). Table 1 below details the preferred measurement values of h1, h2 and h3 for the receiver (44) of FIG. 3 and for the receiver (44′) of FIG. 5. Table 1 also shows, as a percentage, the relative length of each section h1, h2 and h3 against the overall length of the expansion chamber (50, 150) of both embodiments.

TABLE 1
Comparison of Expansion Chamber Dimensions between Prior
Art Receiver and Receiver of the Current Invention

| | | Percentage of | | Percentage of |
|---|---|---|---|---|
| Measure- | Receiver | Overall Length | Receiver | Overall Length |
| ment | 44 of | of Expansion | 44′ of | of Expansion |
| Length | | Chamber | | Chamber |
| h1 | 0.975 mm | 19.9% | 2.95 mm | 27.7% |
| h2 | 0.975 mm | 19.9% | 4.75 mm | 44.6% |
| h3 | 2.95 mm | 60.2% | 2.95 mm | 27.7% |

As will be noted, the lower portion (52) and (152) of both receivers (44) and (44′) are constant and provide a constant engagement or coupling of receivers (44 or 44′) with the upper nozzle (20) of the MDI valve. The modification of the receiver design according to the present invention is effected in the upper portion (154) of the receiver (44′) whose dimensions are significantly altered as compared to the prior art. In the modified receiver design, both the h1 and h2 length are significantly increased over the prior art and the emission orifice (158) is no longer disposed halfway along the length of the flat surface (156). Increases in the h1 and h2 lengths as between the embodiments in FIG. 5 and FIG. 3 also necessitate overall increase in length of the receiver (44′).

The increases in dimensions of the expansion chamber (150) have two significant effects. Firstly, the creation of a greater volume of the expansion chamber as compared to the prior art device will serve to effect greater pressure reduction of the pharmaceutical composition injected therein (as a result of the permissible expansion), whereby the increase in length h1 and h2 both increase the distance that the propelled material must travel before hitting an end surface (120), which serves to retard the velocities of the pharmaceutical content after emission from the metering valve. Ideally, the ratios of the length h1 to h2 will be in the range of 1 to 1.3 to 1 to 2.5. Particularly, the travel of the emitted product along length h1 is duplicated since the distance travelled by the emitted pharmaceutical product will travel past the orifice (158), strike the end surface (170), and be reflected back towards to the emission orifice (158) before being emitted from the receiver. Thus an increase in the value of h1 results in an effective duplication of the slowing effect achieved by this modified receiver design. The increases in distance travelled by the pharmaceutical product prior to passing through the emission orifice (158) serve to reduce the pressure and velocities of the emitted pharmaceutical materials. As such, the reduction of the velocity of the pharmaceutical material at the point of emission from the valve to that of emission from the emission orifice (158) will have reduced sufficiently so as to correspond to the pressure and velocity associated with emission of similar compositions utilising CFC propellants, so that the spray pattern of the emitted pharmaceutical product from the metered dose inhaler are visually similar between existing CFC MDIs and improved HFA MDIs.

In addition, for existing CFC metered dose inhalers (as shown in FIG. 1) the diameter of the emission orifice (58) is maintained in a range of 0.4 mm to 1.0 mm resultant from the inherent pressures of CFC based products being between 45 psi and 60 psi and known to produce approved dosage emissions in known spray patterns. To ensure that the spray formation of metered dose inhalers utilising HFA propellants have the same expansion on emission from the metered dose inhaler (so that the spray is provided with a narrow root and the plume expanding to its fullest dispersal only after passing from the mouthpiece of the adapter), the modified receiver is provided with an emission orifice having a diameter of between on 0.2 mm and 0.3 mm so as to co-operate with the modified pharmaceutical formulation utilising surfactant concentration greater than 0.015% (which provides enhanced droplet formulation) and the different velocity and pressure of the emitted spray.

Also, whilst the external diameter of the prior art receiver (44′) and the external diameter of the receiver (44) of the current invention are substantially the same, it can be seen that the emission orifice length through the respective side wall of the expansion chambers of each receiver must vary, resultant from the increased wall thickness created by the inclined flat surface (156) of the receiver (44′) as opposed to the receiver (44) of the prior art. Effectively, the length of the emission orifice (158) will be in the range of 0.25 mm to 0.29 mm whereas the emission orifice (58) of the prior art will conventionally lie in the range of 0.8 mm to 1.44 mm.

In this manner, by mechanical modification of the receiver of the adaptor, the inherent emission properties of the pharmaceutical product as between the improved pharmaceutical composition utilising HFA propellant and that used in CFC propellant is minimised by reducing the pressure of the emitted pharmaceutical product in an enlarged expansion chamber of the receiver.

Whilst preferred dimensions of the invention are given in Table 1, it will be appreciated that the length h1 may be varied between 1.5 mm and 5.5 mm with a variation in the h2 dimension of between 1.5 mm and 10.5 mm. It is also preferable to maintain h1 as between 20% and 35% of the overall length of the expansion chamber whereby h2 should be maintained of between 35% and 55% of the overall length of the expansion chamber.

It is to be noted that whilst the current MDI design specifically utilises a receiver design having a cylindrical expansion chamber, the exact cross sectional geometry of such expansion chamber may be varied to other geometrical shapes.

However, the objective in the variance of the expansion chamber (150) of the present invention is to compensate for any increased pressure of the propellant now incurred by utilising HFA propellants (as compared to using CFC propellants) so that the spray emission pattern that is emitted from the emission chamber (160) corresponds substantially to the spray pattern of that emitted from MDIs using conventional CFC propellants. It will be appreciated that the variants in volume of the expansion chamber (150) can be effected by varying lengths and/or width and/or the internal profile of this expansion chamber whereas variants in the length and diameter of the emission orifice will also serve to vary the spray pattern emitted from the receiver. Thus, the innovative aspect of this modification is in recognising the need to change the volume of such expansion chamber and the profile of the emission orifice so as to maintain continuity of the medicament product emitted from the emission chamber. This can be achieved through trial and error through visual measurement of the emitted spray patterns for different expansion chamber parameters or can alternatively be effected by determining the formulation pressure of a metered dose of formulation within the expansion chamber for conventional (CFC propellant) product and replicating such pressure measurement for a metered dose of the formulation with HFA propellant in an improved expansion chamber. This provides a means of varying the pressure of the emitted formulation between the metered valve and the emission orifice so as to ensure the correct dispersal of the medicament product to reflect that of previously approved MDIs utilising CFC propellants. It will also be appreciated that should alternative propellants be determined which operate at a lower internal pressure, then the volume of the expansion chamber could conversely be reduced so as to compensate for the reduction in pressure so that the emitted spray reflects the spray patterns of MDIs utilising CFC propellants.

Whilst the preferred embodiment of the current invention is specifically directed to an MDI for administration of the drug beclamethasone dipropionate, it is equally applicable to other drug compositions and notably salbutamol sulphate.

The contents of each of the references cited herein, including the contents of the references cited within the primary references, are herein incorporated by reference in their entirety.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

1. A metered dose inhaler comprising: an aerosol canister containing an aerosol formulation, said canister being in communication via a metered valve with an elongated expansion chamber, said expansion chamber including an emission orifice;
wherein the aerosol formulation comprises: a drug; one or more hydrofluoroalkane propellants; a cosolvent having higher polarity than the propellant(s) and a surfactant; and
wherein said propellant has a pressure of between 70 and 85 psi; and said emission orifice of the inhaler is disposed at a position between 65% and 75% along the length of said elongated expansion chamber in a direction away from said metered valve.

2. A metered dose inhaler as claimed in claim 1, wherein the said emission orifice has a diameter of between 0.2 mm and 0.3 mm.

3. A metered dose inhaler as claimed in claim 1, wherein the chamber length is between 5.95 mm and 18.95 mm in total length.

4. A metered dose inhaler as claimed in claim 1, wherein the propellant comprises one of either 1,1,1,2-tetrafluoroethane (HFA 134a) or 1,1,1,2,3,3,3,-hepafluoropropane (HFA134a) or a mixture thereof.

5. A metered dose inhaler as claimed in claim 1, wherein the surfactant is present in an amount at least 0.01% by weight of said formulation.

6. A metered dose inhaler as claimed in claim 1, wherein the said expansion chamber is tapered in a longitudinal direction such that its cross sectional area at a remote internal end of such expansion chamber is less than 50% of the cross sectional area of the largest cross sectional area of such expansion chamber.

7. A metered dose inhaler as claimed in claim 6, wherein the said remote end is less than 30% of such cross sectional area.

8. A metered dose inhaler as claimed in claim 1, wherein the said expansion chamber is substantially cylindrical at a first end which cylindrical section extends for at least 25% of the overall length of the expansion chamber.

9. A metered dose inhaler as claimed in claim 1, wherein the said chamber comprises a tapered section comprising an inclined inner wall of said chamber, which wall is inclined at an angular range of between 5° and 35° relative to an axis of said cylindrical chamber, said wall being flat, and extending between 65% and 75% of the total length of the chamber.

10. A metered dose inhaler as claimed in claim 1, comprising an aerosol canister containing 15-20 g of said formulation.

11. A metered dose inhaler as claimed in claim 10, wherein the inhaler is arranged to dispense 10-300 mcg of said drug on each actuation of the inhaler.

12. A metered dose inhaler as claimed in claim 1, wherein the inhaler has anodised interior surfaces.

13. A metered dose inhaler as claimed in claim 1, wherein the surfactant is present in an amount up to 0.1% by weight of the formulation.

14. A metered dose inhaler as claimed in claim 1, wherein the surfactant is present in an amount between 0.01-0.02% by weight; optionally wherein the surfactant is present in an amount 0.015% by weight, of said formulation.

15. A metered dose inhaler as claimed in claim 1, wherein the wt/wt ratio of said surfactant to said drug is in the range 0.05-0.5.

16. A metered dose inhaler as claimed in claim 1, wherein the surfactant and said drug together constitute 0.03-0.5% by weight of said formulation.

17. A metered dose inhaler as claimed in claim 1, wherein the drug is present in an amount between 0.01-0.5% by weight.

18. A metered dose inhaler as claimed in claim 1, wherein the wt/wt ration of said cosolvent to said propellant is in the range 0.09-0.1%.

19. A metered dose inhaler as claimed in any one of claims 1, wherein the formulation comprises 0.015-0.089% by weight of said surfactant; 0.014-0.445% by weight of said drug; 0.856-25.68% by weight of said cosolvent; and at least 73.786% by weight of 1,1,1,2-tetrafluoroethane.

20. A metered dose inhaler as claimed in claim 1, wherein the surfactant comprises any of ethyl oleate, sorbitan trioleate, or isopropyl myristate or mixture thereof.
